(19) United States
(12) Patent Application Publication
(10) Pub. No.: US 2008/0004535 A1
(43) Pub. Date: Jan. 3, 2008

(54) IMPLANTABLE MEDICAL DEVICE WITH SENSING ELECTRODES

(76) Inventor: Karel F.A.A. Smits, Munstergeleen (NL)

Correspondence Address: MEDTRONIC, INC., 710 MEDTRONIC PARKWAY NE, MINNEAPOLIS, MN, 55432-9924, US

(21) Appl. No.: 11/427,647

(22) Filed: Jun. 29, 2006

(51) Int. Cl. A61B 5/04

(52) U.S. Cl. 600/509

(57) ABSTRACT

An implantable medical device (IMD) with sensing electrodes that are relatively closely spaced subcutaneous electrodes arranged in a subcutaneous electrode array (SEA). The SEA is implemented to enable a leadless orientation-insensitive SEA scheme for receiving ECG signals. The SEA is distributed over the perimeter of the IMD and includes a non-conductive shroud. The shroud can be wider near the SEA electrodes, providing an enhanced signal to noise ratio, and narrower elsewhere to avoid interfering with pacing or defibrillation signals when the case of the medical device is used as an electrode.

### FIELD OF THE INVENTION

The present invention relates to implantable medical devices. Some embodiments are more particularly related to an IMD with a subcutaneous electrode array (SEA).

### BACKGROUND OF THE INVENTION

The detection, analysis and storage of ECG and EGM data are well known in the art. External ECG recording devices are commonly attached to a patient via multiple ECG leads connected to pads arrayed on the patient's body so as to achieve a recording that displays the cardiac waveforms in any one of 12 different vectors. Such external ECG recorders tend to be impractible for ambulatory use. Holter monitors are well known external devices for monitoring ECGs over short periods of time. However, Holter monitors are bulky and require patient compliance, which cannot always be guaranteed. Monitoring can be done using implantable pulse generators such as pacemakers and other heart stimulating devices or devices with leads in the heart for capturing physiologic parameters. However, certain IPGs are better suited for EGM measurement instead of ECG measurement.

IMDs with SEAs are used in the art to measure ECGs, but because of the relatively remote location of these devices and the relatively small distance between electrodes, achieving an acceptable signal to noise ratio can be challenging. This challenge can be even greater if portions of the IMD are uninsulated, reducing potential differentials in electrical potential in tissues adjacent to uninsulated portions of the IMD.

### BRIEF DESCRIPTION OF THE DRAWINGS

FIG. 1 is an illustration of a body-implantable device system in accordance with one embodiment of the invention.

FIG. 2 is a simplified block diagram of an embodiment of IMD circuitry and associated leads that may be employed in the system of FIG. 1 to enable selective therapy delivery and monitoring.

FIG. 3 is a breakaway drawing of an embodiment of an implantable medical device in accordance with the invention.

FIG. 4 is a sectional view of an embodiment of a shroud in accordance with the invention displaying electrical connections of electrodes to hybrid circuitry.

FIG. 5 is a sectional view of an embodiment of a shroud in accordance with the invention prior to its fixation on the periphery of an implantable medical device.

FIG. 6 is a plan view of a implantable medical device in accordance with the invention.

FIG. 7 is a side view of the implantable medical device shown in FIG. 6.

FIG. 8 is a breakaway of a cross section of the implantable medical device of FIG. 6, wherein the cross section is taken as indicated by line “B-B” in FIG. 6.

FIG. 9 is a cross section of the implantable medical device of FIG. 6.

### DETAILED DESCRIPTION OF THE DRAWINGS

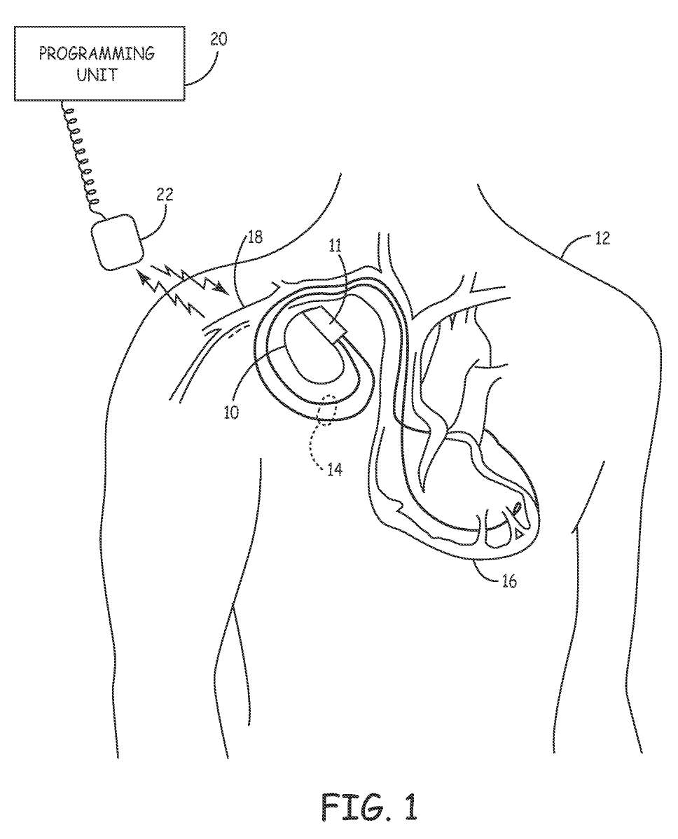

FIG. 1 is an illustration of an implantable medical device system adapted for use in accordance with the present invention. The medical device system shown in FIG. 1 includes an implantable device 10 that has been implanted in a patient 12. In accordance with conventional practice in the art, device 10 is housed within a hermetically sealed, biologically inert outer casing, which may itself be conductive so as to serve as an indifferent electrode in a device's pacing/sensing circuit or a defibrillator's sense/shock circuit. One or more leads, collectively identified with reference numeral 14 in FIG. 1 are electrically coupled to device 10 in a conventional manner and extend into the patient's heart 16 via a vein 18. These leads may generally be referred to as intravenous leads. Disposed generally near the distal end of leads 14 are one or more exposed conductive electrodes for receiving electrical cardiac signals and/or for delivering electrical pacing stimuli to heart 16. As will be appreciated by those of ordinary skill in the art, leads 14 may be implanted with the distal end situated in the atrium and/or ventricle of heart 16. Some devices in accordance with the invention may include subcutaneous defibrillation leads in addition to or instead of intravenous leads. Such a lead may, for example, be oriented under the skin at the patient's back. In such an embodiment the casing of the device may be an active electrode that, in conjunction with such a subcutaneous lead, can deliver a defibrillation shock if needed.

Although the present invention will be described herein in one embodiment which includes a pacemaker/defibrillator, those of ordinary skill in the art having the benefit of the present disclosure will appreciate that the present invention may be advantageously practiced in connection with numerous other types of implantable medical device systems, and indeed in any application in which it is desirable to provide a communication link between two physically separated components, such as may occur during transtelephonic monitoring.

Also depicted in FIG. 1 is an external programming unit 20 in accordance with an embodiment of the invention. The unit 20 may be used for non-invasive communication with implanted device 10 via uplink and downlink communication channels, to be hereinafter described in further detail. Associated with programming unit 20 is a programming head 22, in accordance with conventional medical device programming systems, for facilitating two-way communication between implanted device 10 and programmer 20. In many known implantable device systems, a programming head such as that depicted in FIG. 1 is positioned on the patient's body over the implant site of the device (usually within 2- to 3-inches of skin contact), such that one or more antennae within the head can send RF signals to, and receive RF signals from, an antenna disposed within the hermetic enclosure of the implanted device or disposed within the connector block of the device, in accordance with common practice in the art.

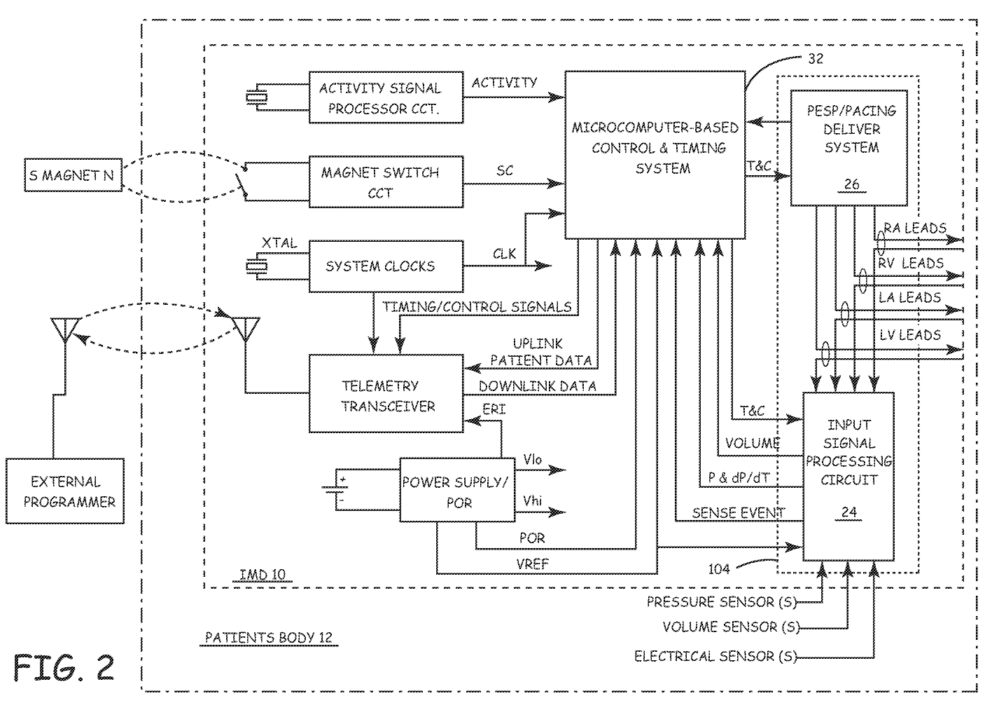

FIG. 2 depicts a system architecture of an exemplary multi-chamber monitor/sensor device 10 implanted into a patient's body 12 that provides delivery of a therapy and/or physiologic input signal processing. The typical multi-chamber monitor/sensor device 10 has a system architecture that is constructed about a microcomputer-based control and timing system 32 which varies in sophistication and complexity depending upon the type and functional features incorporated therein. The functions of microcomputer-based multi-chamber monitor/sensor control and timing system 32 are controlled by firmware and programmed software algorithms stored in RAM and ROM including PROM and EEPROM and are carried out using a CPU or ALU of a typical microprocessor core architecture.

The therapy delivery system 26 can be configured to include circuitry for delivering cardioversion/defibrillation shocks and/or cardiac pacing pulses delivered to the heart or cardiomyostimulation to a skeletal muscle wrapped about the heart. Alternately, the therapy delivery system 26 can be configured as a drug pump for delivering drugs into the heart to alleviate heart failure or to operate an implantable heart assist device or pump implanted in patients awaiting a heart transplant operation.

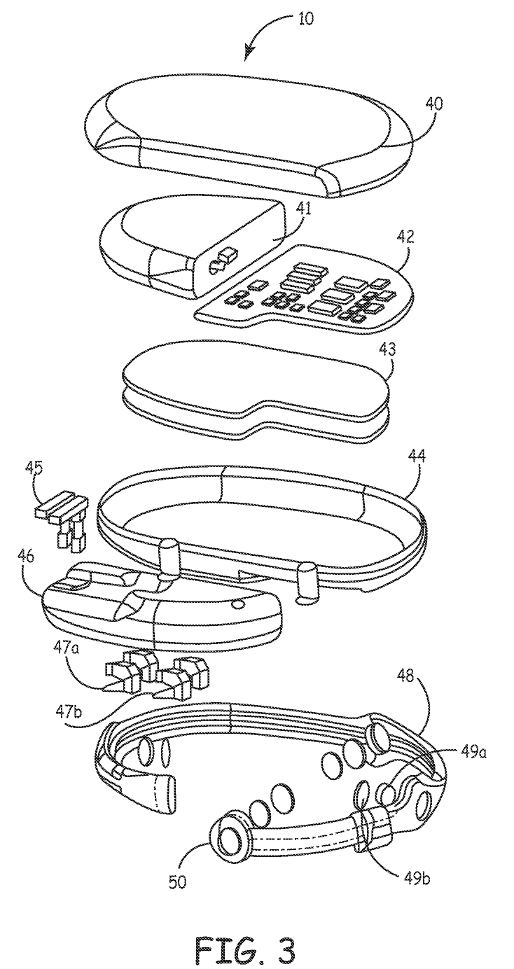

The input signal processing circuit 24 includes at least one physiologic sensor signal processing channel for sensing and processing a sensor derived signal from a physiologic sensor located on the surface of the device 10, in relation to a heart chamber, or elsewhere in the body. Examples illustrated in FIG. 3 include electrical, pressure, and volume sensors, but could include other physiologic or hemodynamic sensors. Physically, the connections between leads 14 and the various internal components of device 10 are facilitated by means of a conventional connector block assembly 11, shown in FIG. 1. Electrically, the coupling of the conductors of leads and internal electrical components of pulse generator 10 may be facilitated by means of a lead interface circuit which functions, in a multiplexer-like manner, to selectively and dynamically establish necessary connections between various conductors in leads 14, and individual electrical components of pulse generator 10, as would be familiar to those of ordinary skill in the art. For the sake of clarity, the specific connections between leads 14 and the various components of pulse generator 10 are not shown in FIG. 2, although it will be clear to those of ordinary skill in the art that, for example, leads 14 will necessarily be coupled, either directly or indirectly, to sense amplifier circuitry 24 and stimulating pulse output circuit 26, in accordance with common practice, such that cardiac electrical signals may be conveyed to sensing circuitry 24, and such that stimulating pulses may be delivered to cardiac tissue, via leads 14. Also not shown in FIG. 2 is the protection circuitry commonly included in implanted devices to protect, for example, the sensing circuitry of the device from high voltage stimulating pulses.

As previously noted, implantable medical device 10 includes central processing unit 32 which may be an off-the-shelf programmable microprocessor or micro controller, but in the present embodiment is a custom integrated circuit. Although detailed connections between CPU 32 and other components of implantable medical device 10 are not shown in FIG. 2, it will be apparent to those of ordinary skill in the art that CPU 32 functions to control the timed operation of stimulating pulse output circuit 26 and sense amplifier circuit 24. It is believed that those of ordinary skill in the art will be familiar with such an operative arrangement.

It is to be understood that the various components of device 10 depicted in FIG. 2 are powered by means of a battery (not shown) which is contained within the enclosure of device 10, in accordance with common practice in the art. In some embodiments in accordance with the invention the enclosure or casing may be hermetically sealed. For the sake of clarity in the Figures, the battery and the connections between it and the other components of device 10 are not shown.

Stimulating pulse output circuit 26, which functions to generate cardiac stimuli under control of signals issued by CPU 32, may be, for example, of the type disclosed in U.S. Pat. No. 4,476,868 to Thompson, entitled “Body Stimulator Output Circuit,” which patent is hereby incorporated by reference herein in relevant part. Again, however, it is believed that those of ordinary skill in the art could select from among many various types of prior art pacing and/or defibrillation output circuits that would be suitable for the purposes of practicing the present invention.

Sense amplifier circuit 24, which is of conventional design, functions to receive electrical cardiac signals from electrodes 49b (described below) and leads 14 and to process such signals to derive event signals reflecting the occurrence of specific cardiac electrical events, including atrial contractions (P-waves) and ventricular contractions (R-waves). CPU provides these event-indicating signals to CPU 32 for use in, among other things, controlling the synchronous stimulating operations or defibrillation operations of device 10 in accordance with common practice in the art. In addition, these event indicating signals may be communicated, via uplink transmission, to external programming unit 20 for visual display to a physician or clinician.

Those of ordinary skill in the art will appreciate that device 10 may include numerous other components and subsystems, for example, activity sensors and associated circuitry. The presence or absence of such additional components in device 10, however, does not affect the scope of the claims appended hereto.

FIG. 3 is a breakaway drawing of an embodiment of an implantable medical 10 in accordance with the invention. The outer casing of the device 10 is composed of right casing 40 and left casing 44. Left casing 44 of this embodiment also has a feedthrough assembly through which wires electrically connecting the lead contacts 47a and 47b to hybrid circuitry 42 are passed. Power to circuitry 42 is provided by battery 41. Pacing, defibrillation, and/or sensing leads (not shown) are inserted into lead connector module 46 so that the portion of the lead that leads to the lead ring electrode makes electrical contact with lead contact 47a and lead tip (distal) electrode makes electrical contact with lead contact 47b when lead fastener 46 is turned to its closed position.

Continuing with FIG. 3, the mechanical portion of this embodiment consists of a shroud 48 that is affixed circumferentially around the perimeter of the implantable medical device. In order to increase electrical isolation of the electrodes 49b from the uninsulated casing 40, 44 without impeding the defibrillation current flow to the casing 40, 44 when the device 10 is utilized as an electrode (active can). In some embodiments of the invention, a circumferentially oriented shroud 48 has a length generally parallel to the perimeter of the device and a width generally perpendicular to the length. Embodiments of such a shroud may be approximately 2-10 times as wide proximate to the electrodes 49b as compared to the balance of the shroud. In some embodiments this relatively wider region may be generally round. In other embodiments the relatively wide region may be elliptical with the longer radius oriented parallel to the width of the shroud. This configuration may advantageously improve the isolation of the electrodes 49b from the casing 40, 44 without impeding the current flow through the casing 40, 44 electrode. This is true because the isopotential lines for the cardiac signals that are being measured by the electrodes 49b are closer together where the curvature is greater, as it is across the width of the shroud and the sensed differential is more easy to undermine if the width is not insulated.

In other embodiments in accordance with the invention, insulating regions of various sizes and shapes may be used. These regions may be contiguous as is shroud 48 or there may be two or more separate regions each associated with one or more sensing electrodes 49b

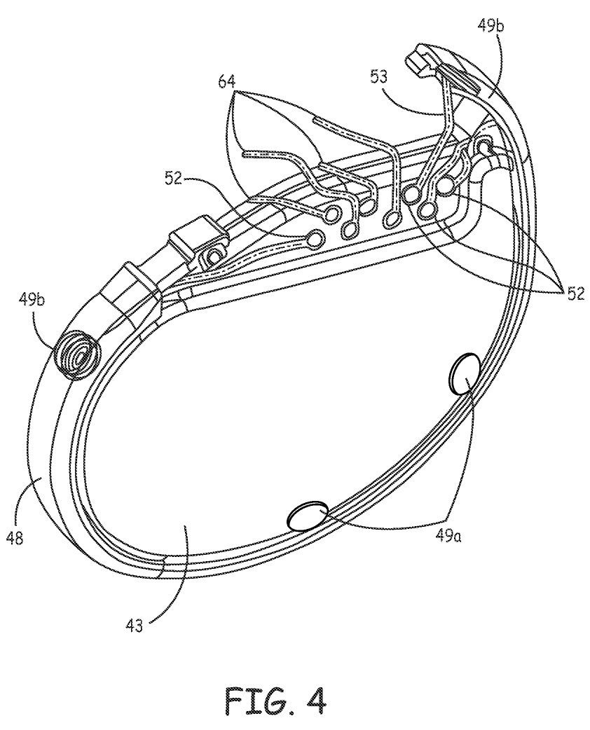

In one embodiment of the present invention, there are four recessed openings 50. A cup 49a with one of the electrodes 49b is fitted into each recessed opening. Into each of recessed openings 50 is placed an electrode such as an electrode that, in conjunction with other paired electrodes detect cardiac depolarizations. These electrical signals are passed to electrode 49b that is electrically connected to hybrid circuitry 42 via insulated wires running on the inner portion of shroud 48 (see FIG. 4 for details). Electrodes 49b may include various types of electrodes with different configurations, such as flat, circular, coiled, concave/convex, or other geometric shapes.

Many, if not all, previous versions of implantable medical devices having sensing electrodes similar to electrodes 49b include insulated cases that generally do not affect the voltage differentials in the tissue proximate to the device. Embodiments of devices in accordance with the current invention may have uninsulated casings 40, 44 that act as electrodes in the delivery of pacing pulses or defibrillation shocks. The insulating shroud 48 in such a device may be configured to optimize isolation of the electrodes 49b from the casing 40, 44 and impedance of the pacing and/or defibrillation current. In other words, the insulation shroud 48 or region on the case 40,44 is more effective as it increases in size, but increasing the size of the insulating shroud or region may impede the electrical current from the active casing electrode.

FIG. 4 is a sectional view of shroud 48 displaying electrical connections of the electrodes to the hybrid circuitry surrounded by insulators 43. Shroud 48 displays recessed cups 49a and electrical contacts 52 all of which are connected to the hybrid circuitry (not shown) via tubular wiring 53. Tubular wiring 53 is connected to electrode contacts 52 located on upper portion of the board holding the hybrid circuitry. Other contacts 64 electrically connect the leads to the hybrid circuitry.

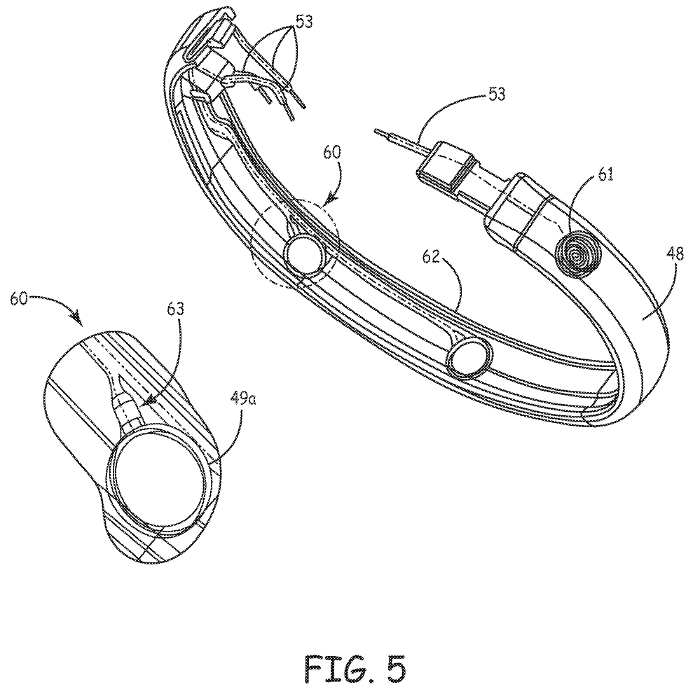

FIG. 5 is a sectional view of shroud 48, prior to its fixation on the periphery of an implantable device 10. Detail 60 shows the bottom of recessed cup 49a into which an electrode contact (not shown) is placed. In one embodiment of the present invention, protruding end of coiled electrode 61 is placed into insulating connector 63 that is welded to tubular wiring 53. Tubular material wiring runs through channels 62 formed on the inside of the shroud 48.

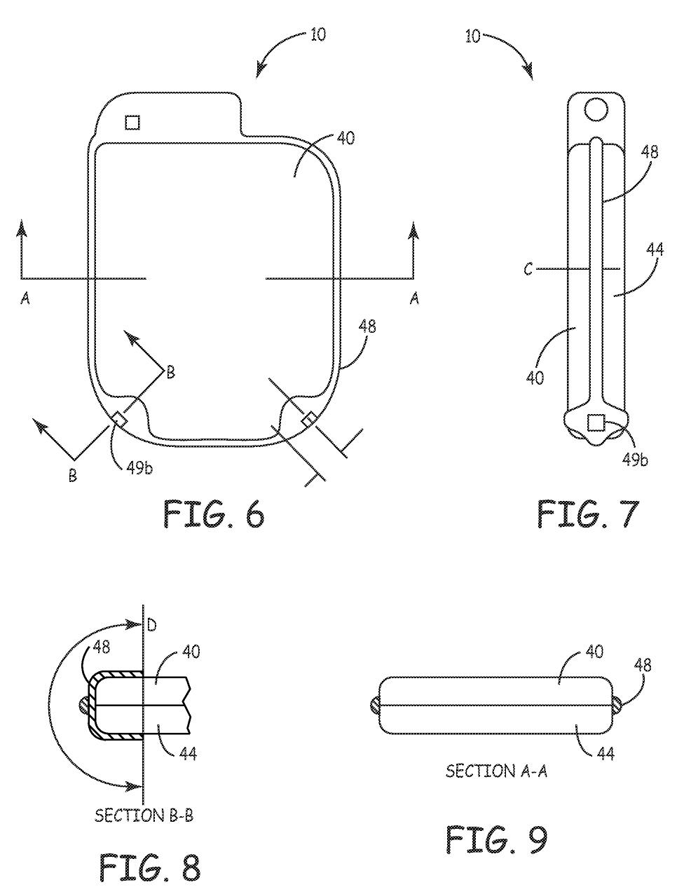

FIG. 6 is a plan view of an implantable medical device in accordance with an embodiment of the present invention. The device 10 shown includes a casing 40, a shroud 48, and an electrode 49b. The shroud 48 is wider proximate the electrode 49b, and in fact in this embodiment extends substantially over the generally flat surface of casing 40. In this way the shroud 48 improves electrical isolation of the electrode 49b.

FIG. 7 is a side view of the implantable medical device shown in FIG. 6. Shown is a device 10 having a casing 40, 44, a shroud 48, and an electrode 49b. The shroud 48 is larger proximate the electrode 49b and narrower away from the electrode as shown at dimension “C.”

FIG. 8 is a breakaway of a cross section of the implantable medical device of FIG. 7, wherein the cross section is taken as indicated by line “B-B” in FIG. 6. Shown is the casing 40, 44, an electrode 49b, and the shroud 48. The shroud in this area is wider at dimension “D” than it is at dimension “C” shown in the previous FIG. 8. In some embodiments of the invention, dimension “D” is 2 to 8 times as wide as dimension “C.”

FIG. 9 is a cross section of the implantable medical device of FIG. 6. Shown is the casing 40, 44 and the shroud 48. As can be seen in this figure, the shroud 48 of this embodiment does not cover even the peripheral edges of the casing 40, 44 at the point at which the cross section is taken. This reduced coverage of the casing 40, 44 lowers impedance when the casing is used as an active electrode. The combination of the wider shroud 48 proximate the electrode 49b shown in FIGS. 6-8 and the narrower shroud on the balance of the device shown in FIGS. 6, 7, and 9 provides for improved isolation of sense electrodes 49b from the uninsulated portions of casing 40, 44 while mitigating the impact of shroud 48 on the ability to use casing 40, 44 as an electrode in pacing and defibrillation applications.

Implantable medical devices employing subcutaneous electrode arrays may be designed to maximize the distance between electrodes. In general, as the number of electrodes increase, the magnitude of the detected cardiac signal increases. Selection of the number and location of electrodes is a matter that is well known in the art and considerations may include capture certainty, cost, device complexity, and others known in the art.

It will be appreciated by those skilled in the art that while the invention has been described above in connection with particular embodiments and examples, the invention is not necessarily so limited and that numerous other embodiments, examples, uses, modifications and departures from the embodiments, examples and uses may be made without departing from the inventive concepts.

What is claimed is:

1.-20. (canceled)

21. A cardiac data acquisition system comprising:
a. an implantable medical device having a case;
b. an insulating shroud on the case;
c. two or more electrodes disposed on the shroud, the insulating shroud being relatively larger at portions proximate to the electrodes; and
d. signal processing circuitry disposed inside the case, the signal processing circuitry being electrically coupled to the electrodes to detect cardiac signals.

22. The cardiac data acquisition system of claim 21, wherein the insulating shroud comprises a band around the medical device, the band being relatively wider proximate to the electrodes.

23. The cardiac data acquisition system of claim 21, wherein the insulating shroud comprises a band around the case, the band being relatively narrower at locations distal to the electrodes.

24. The cardiac data acquisition system of claim 21, wherein the insulating shroud insulates the electrodes from the case.

25. The cardiac data acquisition system of claim 21, wherein the insulating shroud surrounds a perimeter of each electrode.

26. The cardiac data acquisition system of claim 21, wherein the insulating shroud includes recesses into which electrodes may be embedded.

27. The cardiac data acquisition system of claim 21, wherein the case provides an active electrode for the implantable medical device.

28. An apparatus for leadless acquisition of electrocardiographic data comprising:
a. an implantable device case having insulative and conductive regions on the surface of the case, the conductive regions functioning as an indifferent electrode for pacing or defibrillation; and
b. sensing electrodes capable of sensing cardiac depolarization, the sensing electrodes disposed on the insulative regions, the insulative regions increasing voltage differentials between the sensing electrodes and the conductive regions without substantially degrading use of the conductive regions for defibrillation or pacing.

29. The apparatus of claim 28, wherein the insulative regions comprise an insulative shroud insulating the sensing electrodes from the conductive regions.

30. The apparatus of claim 29, wherein the insulative shroud includes relatively wider regions proximate the sensing electrodes and relatively narrower regions distal from the sensing electrodes.

31. The apparatus of claim 29, wherein the case includes a peripheral edge surface, the insulative shroud being disposed along the peripheral edge surface.

32. The apparatus of claim 31, wherein the case includes a peripheral edge surface, the electrodes being disposed along the peripheral edge surface of the case at locations providing maximal electrode spacing.

33. The apparatus of claim 28, wherein the sensing electrodes are disposed with generally maximum spacing therebetween.

34. The apparatus of claim 28, further comprising one or more sense amplifiers each having terminals, the electrodes being sequentially coupled in one or more pairs to the terminals.

35. The apparatus of claim 34, wherein pairs of the sensing electrodes provide vectors over which to sense cardiac signals, and further comprising means to analyze cardiac signals sensed by the pairs of electrodes to determine the one of the sensing vectors that provides the largest cardiac signal.

36. An insulating shroud mechanism for an implantable medical device comprising:
a. a shroud configured to be attached to the perimeter of a generally planar implantable medical device, the shroud having a length generally oriented around the perimeter and a width generally perpendicular to the length;
b. at least one site on the shroud configured to house an electrode, the shroud being wider proximate the at least one site than along the balance of the length of the shroud.

37. The insulating shroud mechanism of claim 36, wherein the at least one site includes a recess for housing an electrode.

38. The insulating shroud mechanism of claim 36, wherein the width of the shroud proximate the at least one site is between two and eight times the width of the shroud along the balance of the length of the shroud.

39. The insulating shroud mechanism of claim 36, wherein the shroud includes a channel extending along the length of an inside face of the shroud, the channel being configured to house tubular wiring.

40. The insulating shroud of claim 36, wherein the sites have locations providing maximum spacing.
